(19) United States
(12) Patent Application Publication
(10) Pub. No.: US 2026/0240826 A1
(43) Pub. Date: Aug. 20, 2026

(54) COMPOSITIONS FOR PD-L1 INHIBITION AND METHODS OF USE THEREOF

(71) Applicant: ARIZONA BOARD OF REGENTS ON BEHALF OF THE UNIVERSITY OF ARIZONA, Tucson, AZ (US)

(72) Inventors: Sally E. Dickinson, Tucson, AZ (US); Georg Wondrak, Tucson, AZ (US); Clara N. Curiel-Lewandrowski, Tucson, AZ (US)

(21) Appl. No.: 19/162,362

(22) PCT Filed: Mar. 6, 2024
(86) PCT No.: PCT/US24/18709

§ 371 Date: Sep. 4, 2025

Related U.S. Application Data
(60) Provisional application No. 63/488,621, filed on Mar. 6, 2023.

(51) Int. Cl. A61K 31/44; A61K 8/49; A61P 35/00; A61Q 17/04

(52) U.S. Cl. CPC A61K 31/44; A61K 8/4926; A61P 35/00; A61Q 17/04

(57) ABSTRACT

One of three newly diagnosed cancers is skin cancer, making skin cancer the most common malignancy worldwide. Currently, no topically applied pharmacological interventions inhibit PD-L1 to protect against skin cancer and other solar radiation-induced skin damage. Thus, described herein are topical compositions comprising a small molecule antagonist of programmed death-ligand 1 (PD-L1), e.g., BSM-202, and methods of use thereof for skin protection.

### CROSS-REFERENCE TO RELATED APPLICATIONS

This application claims benefit of U.S. Provisional Application No. 63/488,621 filed Mar. 6, 2023, the specification of which is incorporated herein in their entirety by reference.

### STATEMENT REGARDING FEDERALLY SPONSORED RESEARCH OR DEVELOPMENT

This invention was made with government support under Grant No. CA229112 awarded by National Institutes of Health. The government has certain rights in the invention.

### FIELD OF THE INVENTION

The present invention features compositions comprising a small molecule antagonist of programmed death-ligand 1 (PD-L1) for skin protection and methods of use thereof.

### BACKGROUND OF THE INVENTION

Non-melanoma skin cancer, primarily comprised of cutaneous squamous cell carcinoma (cSCC) and basal cell carcinoma (BCC), is the most common malignancy worldwide. Diagnosis and treatment of these keratinocytic neoplasms result in healthcare costs of $8.1 billion/year in the United States. Cutaneous exposure to solar ultraviolet (UV) radiation is the lead causative factor in skin carcinogenesis, and inflammatory dysregulation is an accepted key mechanism underlying the detrimental effects of acute and chronic UV exposure. Thus, the present invention features novel methods to prevent and treat cSCCs, which account for 20% of all non-melanoma skin cancers and kills more than 15,000 people a year.

Programmed death-ligand 1 (PD-L1, also known as CD274 or B7-H1) is a transmembrane protein that regulates T-cell responses. Binding of PD-L1 to its receptor, programmed cell death protein-1 (PD-1), on the surface of T cells suppresses T cell proliferation and activity, an interaction often referred to as an immune checkpoint. The PD-1/PD-L1 interaction as a negative regulator of immune cell activation (and, therefore, an effector of immune evasion) is now an established target in cancer therapy.

Expression of PD-1 is typically restricted to immune cells (including T cells, B cells, and natural killer cells), and immunotherapies using systemic monoclonal antibodies against PD-1 have been approved for many advanced malignancies, including cSCC, BCC, and melanoma. In contrast, PD-L1 is either basally expressed or inducible in most cells of the body. Overexpression of PD-L1 in cSCC is common, and there is a correlation between increased PD-L1 expression and advanced clinical risk assessment in these tumors. In addition to PD-1-directed interventions, the blockade of PD-L1 by immunotherapeutics is now used clinically in many types of cancer (including melanoma and BCC) and is being studied for use as adjuvant therapy for the treatment of cSCC.

In normal skin, PD-L1 expression has been studied extensively in mouse models and only recently in human samples. The PD1/PD-L1 pathway is critical for regulating skin inflammation, and PD-L1 on keratinocytes has been shown to regulate autoimmunity. Transgenic overexpression of PD-L1 in mouse keratinocytes reduces acute skin inflammatory responses yet increases the rates of skin tumorigenesis and risk of death after skin stimulation with a chemical carcinogen.

While little is known about the overall trajectory of PD-L1 expression during the development of cSCC, there is evidence that this ligand may have potential as a target for skin cancer prevention strategies. Recent evidence indicates that exposure to acute UV via solar-simulated light (SSL) in human and mouse skin causes significant upregulation of PD-L1 protein in epidermal keratinocytes from its low baseline expression levels. This suggests that a primary environmental causative factor driving skin carcinogenesis, UV light, is sufficient to modulate PD-L1 in the epidermis. Therefore, the present invention features methods for topical application of a PD-L1-specific small molecule pharmacological inhibitor, BMS-202, that can affect UV-induced stress responses.

### BRIEF SUMMARY OF THE INVENTION

It is an objective of the present invention to provide compositions and methods that protect skin against environmental insult originating from solar radiation (UV), and other toxicants such as pollutants, as specified in the independent claims. Embodiments of the invention are given in the dependent claims. Embodiments of the present invention can be freely combined with each other if they are not mutually exclusive.

The present invention features in vitro as well as in vivo testing on the small molecule BMS-202. BMS-202 application to keratinocytes in culture inhibits UV-induced PD-L1 RNA and protein expression. Notably, exposure to BMS-202 reduced UV-induced stress signaling as measured by AP-1 luciferase assay in cultured keratinocytes. Topical application of BMS-202 to immunocompetent SKH-1 mouse skin resulted in significant inhibition of SSL-induced epidermal PD-L1 as determined by immunoblot analysis. NanoString nCounter Pathway™ transcriptomic analysis of full-thickness SKH-1 mouse skin shows strong inhibition of SSL-induced inflammatory responses, chemokine activity, innate immune response, and NF-κB activation in BMS-202 treated samples compared to SSL-only controls. These results indicate early intervention against PD-L1 expression/activity could be a viable target for skin cancer photochemoprevention. Topical application of small molecule PD-L1 inhibitors such as BMS-202 may provide novel treatment options for populations at high risk for cSCC.

In some embodiments, the present invention features a method of preventing and reversing damage caused by solar radiation of skin, comprising contacting the skin with a therapeutically effective amount of a composition comprising a small molecule antagonist of programmed death-ligand 1 (PD-L1), thereby protecting the skin from damage caused by solar radiation. In other embodiments, the present invention features a method of preventing and reversing damage caused by solar radiation of skin, comprising contacting the skin with a therapeutically effective amount of a topical composition comprising a small molecule antagonist of programmed death-ligand 1 (PD-L1), thereby protecting the skin from damage caused by solar radiation. In further embodiments, the present invention features a method of preventing and reversing damage caused by solar radiation of skin, comprising contacting the skin with a therapeutically effective amount of a topical composition comprising BMS-202 or variants thereof, thereby protecting the skin from damage caused by solar radiation. The BSM-202 variants herein may be optimized for skin residence time and minimum systemic availability. Compositions herein may be in the form of a lotion, a cream, a balm, an ointment, a gel, a paste, a spray, a patch, or a solution.

In some embodiments, the present invention features a method of suppressing immunotoxic effects caused by solar radiation in a subject in need thereof, the method comprising administering a therapeutically effective amount of a small molecule antagonist of programmed death-ligand 1 (PD-L1) to the subject. In some embodiments, the small molecule antagonist is administered topically. The small molecule antagonist comprises BMS-202 or variants thereof that have been optimized for skin residence time and minimum systemic availability.

In some embodiments, the present invention features a method of preventing or treating skin cancer in a subject in need thereof, the method comprising administering a therapeutically effective amount of a composition comprising a small molecule antagonist of programmed death-ligand 1 (PD-L1). In some embodiments, the present invention features a method of preventing or treating skin cancer in a subject in need thereof, the method comprising topically administering a therapeutically effective amount of a composition comprising a small molecule antagonist of programmed death-ligand 1 (PD-L1). In certain embodiment, the present invention features a method of preventing or treating skin cancer in a subject in need thereof, the method comprising topically administering a therapeutically effective amount of a composition comprising a small molecule antagonist of programmed death-ligand 1 (PD-L1) comprising BMS-202 or variants thereof. The variants of BMS-202 may be optimized for skin residence time and minimum systemic availability. In some embodiments, the composition is in the form of a lotion, a cream, a balm, an ointment, a gel, a paste, a spray, a patch, or a solution. Non-limiting examples of skin cancer may include cutaneous squamous cell carcinoma (cSCC) and basal cell carcinoma (BCC).

In some embodiments, the present invention features a topical composition for skin protection, the composition comprising a small molecule antagonist of programmed death-ligand 1 (PD-L1). In other embodiments, the present invention features a topical composition for skin protection, the composition comprising a small molecule antagonist of programmed death-ligand 1 (PD-L1) comprising BMS-202 or derivatives thereof.

One of the unique and inventive technical features of the present invention is the topical use of the small molecule BMS-202 (or variants thereof). Without wishing to limit the invention to any theory or mechanism, it is believed that the technical feature of the present invention advantageously provides for effective delivery to the skin. None of the presently known prior references or work has the unique, inventive technical feature of the present invention.

Moreover, the prior references teach away from the present invention. For example, antibodies are large molecules and can't be effectively delivered to the skin through topical formulations to the skin.

Furthermore, the inventive technical features of the present invention contributed to a surprising result. For example, blocking PD-L1 impacted numerous UV-induced inflammatory signaling pathways, thought previously not to be impacted by PD-L1 modulation.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A, 1B, 1C, and 1D shows PD-L1 expression in human skin increases as a function of cSCC progression and is detectable in sun-damaged versus sun-protected skin. FIG. 1A shows PD-L1 expression from a clinical cohort stratified as normal skin, low-risk cSCC, or high-risk cSCC (IHC; 9 representative donor specimens, bar=100 μm. FIG. 1B shows quantitative analysis of tissue staining (from FIG. 1A), normal skin: n=20, low-risk SCC: n=40, high-risk SCC: n=31 (p ***<0.001, Kruskal-Wallis non-parametric data analysis). FIG. 1C shows PD-L1 expression in sun-damaged skin (IHC; bar=100 μm, 3 representative donors). FIG. 1D shows proteomic (RPPA) analysis of PD-L1 expression in human epidermal samples stratified by sun-damaged status (SP: sun-protected, SD: sun-damaged). Left: waterfall plot depicting epidermal PD-L1 expression per human skin biopsy, right: box-and-whisker depiction indicating median (line in box) with interquartile range as well as maximum and minimum values (p ***<0.001). See methods for clinical details.

FIGS. 2A, 2B, 2C, 2D, and 2E shows topical application of the PD-L1 inhibitor BMS-202 antagonizes SSL-induced stress signaling, inflammatory gene expression and caspase-3 cleavage in SKH-1 mouse skin. FIG. 2A shows the molecular structure of BMS-202. FIG. 2B shows bioluminescent AP-1 reporter mice display SSL-induced inflammatory signaling suppressed by topical BMS-202 (8 mM). Top: Representative transgenic AP-1 luciferase SKH-1 reporter mice. Bottom: Quantification of bioluminescence intensity. All groups (n=3) were treated with vehicle (acetone) or vehicle+BMS-202. FIG. 2C shows BMS-202 suppression of inflammatory signaling in AP-1 luciferase reporter HaCaT cells (representative of 3 independent experiments; 3 experimental replicates per group). FIG. 2D shows gene-specific gene expression analysis by RT-qPCR indicates BMS-202 suppression of SSL-induced genes in SKH-1 mouse skin (n=3 mice/per group). FIG. 2E shows BMS-202 suppression of SSL-induced caspase-3 cleavage representative skin images (left, bar=100 μm), and quantification (right, n=3 mice/group). Bar graphs depict mean±/−S.D (** indicates p<0.001; *** indicates p<0.0001).

FIGS. 3A and 3B shows nanoString nCounter™ expression analysis of SKH-1 mouse skin after SSL exposure with or without topical BMS-202 treatment. FIG. 3A shows the overall heatmap with z-score. FIG. 3B shows a volcano plot depiction. Analysis was performed using three independent murine specimens per group.

FIGS. 4A, 4B, and 4C shows NanoString nCounter™ expression analysis of SKH-1 mouse skin after SSL exposure with or without topical BMS-202 treatment. FIG. 4A shows heatmap of pathway scores: Clustered analysis of statistically significant expression changes as a function of BMS-202 exposure (8 mM; dark gray: low scores, light gray: high scores); scores are displayed on the same scale via a Z-transformation. FIG. 4B shows the overall pathway score analysis: covariate plot. FIG. 4C shows single pathway score analysis (p<0.05). Analysis was performed using three independent murine specimens per group.

FIG. 5 shows a Heatmap depiction of ‘immune response’ pathway expression data: Heatmap of the normalized data with z-score (vehicle+SSL vs BMS-202+SSL), scaled to give all genes equal variance, generated via unsupervised clustering. Analysis was performed using three independent murine specimens per group.

FIG. 6 shows a Heatmap depiction of ‘inflammatory response’ pathway expression data: Heatmap of the normalized data with z-score (vehicle+SSL vs BMS-202+SSL), scaled to give all genes equal variance, generated via unsupervised clustering. Analysis was performed using three independent murine specimens per group.

FIGS. 7A, 7B, 7C, 7D, 7E, 7F, and 7G shows the effects of BMS-202 on SSL-induced PD-L1 upregulation in cultured human keratinocytes and SKH-1 mouse skin. FIG. 7A shows PD-L1 mRNA expression (24 hr after SSL) is suppressed dose-dependently by BMS-202 treatment in HaCaT keratinocytes. Data depict an experiment run in triplicate (and then repeated 2 more times; bars represent average+/−SD). FIG. 7B shows the inhibition of SSL-induced PD-L1 protein upregulation examined by immunoblot analysis after treatment specified in FIG. 7A. FIG. 7C shows the inhibition of SSL-induced PD-L1 protein upregulation in primary epidermal keratinocytes examined as in FIG. 7B. FIG. 7D shows SSL-induced PD-L1 protein levels in treatment naïve SKH-1 mouse epidermal lysates examined by immunoblot analysis. FIG. 7E shows Inhibition of SSL-induced PD-L1 protein upregulation in SKH-1 mouse epidermis due to topical BMS-202 (8 mM) as achieved by pre-(only) or pre- and post-treatment as determined by immunoblot analysis 24 hr post SSL (left). Data depict individual mouse samples on the left (n=3) with bar graph analysis on the right (average+/−SD; representative experiment out of three independent repeats). FIG. 7F shows immunohistochemistry of pre/post-treated SKH-1 skin as in FIG. 7E, stained for PD-L1 (left, bar=100 μm). Bar graph (right) depicts the quantification of epidermal staining. FIG. 7G shows PD-L1 mRNA expression (24 hr after SSL) in SKH-1 mouse skin topically treated with 8 mM BMS-202. In bar graphs, * indicates p<0.05; ** indicates p<0.001.

### DETAILED DESCRIPTION OF THE INVENTION

For purposes of summarizing the disclosure, certain aspects, advantages, and novel features of the disclosure are described herein. It is to be understood that not necessarily all such advantages may be achieved in accordance with any particular embodiments of the disclosure. Thus, the disclosure may be embodied or carried out in a manner that achieves or optimizes one advantage or group of advantages as taught herein without necessarily achieving other advantages as may be taught or suggested herein.

Additionally, although embodiments of the disclosure have been described in detail, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses and obvious modifications and equivalents thereof. Moreover, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein disclosed should not be limited by the particular disclosed embodiments described herein.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

A “subject” is an individual and includes, but is not limited to, a mammal (e.g., a human, horse, pig, rabbit, dog, sheep, goat, non-human primate, cow, cat, guinea pig, or rodent), a fish, a bird, a reptile or an amphibian. The term does not denote a particular age or sex. Thus, adult and newborn subjects, as well as fetuses, whether male or female, are intended to be included. A “patient” is a subject afflicted with a disease or disorder.

The terms “administering” and “administration” refer to methods of providing a pharmaceutical preparation to a subject. Such methods are well known to those skilled in the art and include, but are not limited to, administering the compositions topically, orally, parenterally (e.g., intravenously and subcutaneously), by intramuscular injection, by intraperitoneal injection, intrathecally, transdermally, extracorporeally, or the like.

The term “skin,” when used herein, is in the broad sense meaning the skin of the face, body, feet, neck, etc. The term “topical composition,” as used herein, shall mean the complete product, including the active components, the carrier, and any adjuvants, thickeners, excipients, etc., as described herein, which is applied to a person's skin. Topical compositions for topical administration include but are not limited to, ointments, lotions, creams, gels, drops, suppositories, sprays, patches, liquids, and powders. Conventional carriers, aqueous, powder or oily bases, thickeners, diluents, scents, emulsifiers, dispersing aids or binders, and the like may be necessary or desirable. Generally, in the practice of methods described herein, the topical composition is topically applied to the skin areas, such as that of the face, arms, legs, feet, and hand, at predetermined or as-needed regimen either at intervals by application of a lotion, a cream, a balm, an ointment, a gel, a paste, or a spray.

As used herein, the term “therapeutically effective amount” refers to an amount of a composition effective to treat a condition, disease, or disorder in a subject. In the case of inflammation, the therapeutically effective amount of the present composition may reduce (i.e., slow to some extent and preferably stop) inflammation and/or relieve, to some extent, one or more of the symptoms associated with a disorder or disease. The “therapeutically effective amount” will vary depending on the compound, the condition and its severity, and body factors such as age, weight, etc., of the subject to be treated.

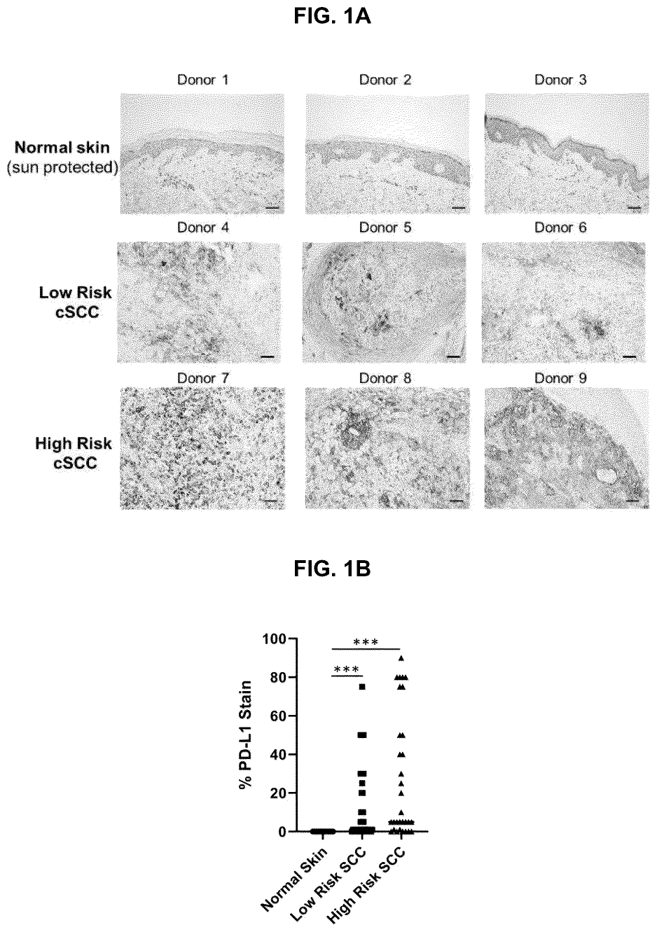

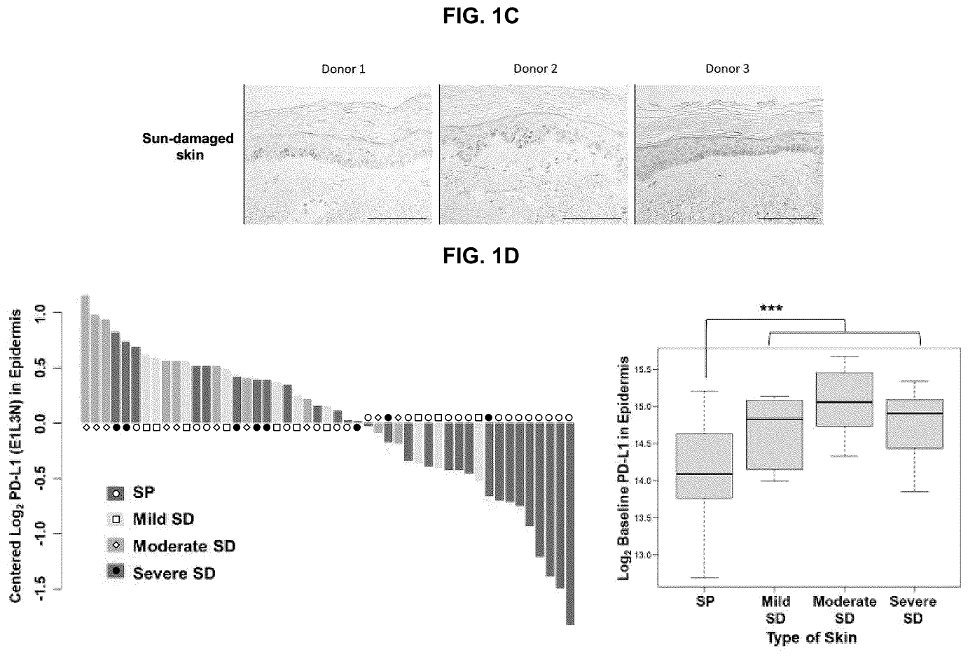

Referring now to FIGS. 1A-7G, the present invention features compositions comprising a small molecule antagonist of programmed death-ligand 1 (PD-L1) and methods of use thereof. Without wishing to limit the invention to a particular theory or mechanism, the small molecule antagonist of PD-L1/PD-1 immune checkpoint signaling described herein (e.g., BMS-202) preserves immune function in skin exposed to toxicants, thereby preventing damage and cancer related to inflammation and immune dysregulation.

The present invention features a method of protecting and reversing damage caused by solar radiation (e.g., ultraviolet (UV) rays) on the skin. The method may comprise contacting the skin with a therapeutically effective amount of a composition comprising a small molecule antagonist of programmed death-ligand 1 (PD-L1), thereby protecting the skin from damage caused by solar radiation (e.g., UV rays). In some embodiments, the method comprises contacting the skin with a therapeutically effective amount of a topical composition comprising BSM-202 or variants thereof, thereby protecting the skin from damage caused by solar radiation (e.g., UV rays). Protecting skin may refer to protection from adverse health consequences and aging due to solar radiation or environmental pollutants.

In some embodiments, the present invention features compositions comprising a small molecule antagonist of programmed death-ligand 1 (PD-L1), e.g., BSM-202 or variants thereof, for protection of the skin against environmental insult originating from solar radiation (UV), and other toxicants such as pollutants. In some embodiments, variants of BSM-202 may include modifications located in the N-acetyl substituent group or, alternatively, in the bi-phenyl group.

The present invention may feature a method of preventing and reversing damage caused by solar radiation (e.g., UV rays) of skin, comprising contacting the skin with a therapeutically effective amount of a composition (e.g., a topical composition) comprising a small molecule antagonist of programmed death-ligand 1 (PD-L1), thereby protecting the skin from damage caused by solar radiation. In some embodiments, the present invention features a method of preventing and reversing damage caused by solar radiation of skin, comprising contacting the skin with a therapeutically effective amount of a topical composition comprising a small molecule antagonist of programmed death-ligand 1 (PD-L1), thereby protecting the skin from damage caused by solar radiation. In other embodiments, the present invention features a method of preventing and reversing damage caused by solar radiation of skin, comprising contacting the skin with a therapeutically effective amount of a topical composition comprising BMS-202 or variants thereof, thereby protecting the skin from damage caused by solar radiation. The variants of BMS-202 are optimized for skin residence time and minimum systemic availability. The aforementioned compositions are in the form of a lotion, a cream, a balm, an ointment, a gel, a paste, a spray, a patch, or a solution.

The methods described herein may reverse and antagonize the damage caused by photodamage, inflammation (dermatitis), autoimmune disease, carcinogenesis and pre-cancerous conditions, photoaging, or a combination thereof.

In some embodiments, inflammation comprises contact dermatitis, e.g., allergic and irritant, psoriasis, etc. In some embodiments, the autoimmune disease comprises vitiligo, scleroderma, lupus, etc. In some embodiments, the pre-cancerous conditions comprise actinic keratosis or dysplastic nevi.

In some embodiments, reversing damage occurs through a process of photorejuvenation or photoimmunoprevention.

In some embodiments, the method further prevents the development of precancerous or cancerous states through early molecular interception.

The present invention may also feature a method of suppressing immunotoxic effects (e.g., extended-expression of immune checkpoints) caused by solar radiation in a subject in need thereof. The method comprises administering a therapeutically effective amount of a small molecule antagonist of programmed death-ligand 1 (PD-L1) to the subject. In some embodiments, the method comprises administering topically a therapeutically effective amount of a small molecule antagonist of programmed death-ligand 1 (PD-L1) to the subject. In other embodiments, the method comprises administering topically a therapeutically effective amount of a small molecule antagonist of programmed death-ligand 1 (PD-L1) comprising BMS-202 or variants thereof to the subject.

The present invention further features a method of protecting skin, comprising contacting the skin with a therapeutically effective amount of a composition comprising a small molecule antagonist of programmed death-ligand 1 (PD-L1), thereby protecting the skin. In some embodiments, the method comprises contacting the skin with a therapeutically effective amount of a topical composition comprising a small molecule antagonist of programmed death-ligand 1 (PD-L1), thereby protecting the skin. In other embodiments, the method comprises contacting the skin with a therapeutically effective amount of a topical composition comprising of a small molecule antagonist of programmed death-ligand 1 (PD-L1) comprising BMS-202 or variants thereof to the subject.

In some embodiments, the damage is caused by solar radiation comprising ultraviolet (UV) rays. In some embodiments, the damage is caused by ionizing and non-ionizing radiation.

In some embodiments, the small molecule comprises BMS-202 or variants thereof. In some embodiments, the variants of BMS-202 are optimized for skin residence time and minimum systemic availability.

In some embodiments, methods described herein protect the skin from toxicants (e.g., pollutants). In some embodiments, methods described herein protect the skin from environmental pollutants. Non-limiting examples of environmental pollutants comprise arsenic, ozone, dioxin, smog, particulate matter (e.g., PM 2.5 nanoparticles (diesel exhaust)), benzo (a) pyrene (BAP), other polyaromatic hydrocarbons, or a combination thereof.

In some embodiments, the compositions described herein are applied topically. For example, the composition may be in the form of a lotion, a cream, a balm, an ointment, a gel, a paste, a patch, a spray, or a solution.

In some embodiments, the methods described herein protect skin against inflammation, immune dysfunction, and cancer (e.g., cutaneous squamous cell carcinoma (cSCC)). In some embodiments, the methods described herein protect skin against atopic dermatitis, inflammation (e.g., contact dermatitis (e.g., allergic and irritant), psoriasis, etc.), and autoimmune disease (e.g., vitiligo, scleroderma, lupus, etc.).

The present invention features a method of preventing or treating skin cancer in a subject in need thereof, the method comprising administering a therapeutically effective amount of a composition comprising a small molecule antagonist of programmed death-ligand 1 (PD-L1). In some embodiments, the method comprises topically administering a therapeutically effective amount of a composition comprising a small molecule antagonist of programmed death-ligand 1 (PD-L1). In other embodiments, the method comprises topically administering a therapeutically effective amount of a composition comprising a small molecule antagonist of programmed death-ligand 1 (PD-L1) comprising BMS-202 or variants thereof. The variants of BMS-202 are optimized for skin residence time and minimum systemic availability. In some embodiments, the composition is in the form of a lotion, a cream, a balm, an ointment, a gel, a paste, a spray, a patch, or a solution. In some embodiments, skin cancer may comprise cutaneous squamous cell carcinoma (cSCC) and basal cell carcinoma (BCC).

The present invention may further feature a topical composition for skin protection, the composition comprising a small molecule antagonist of programmed death-ligand 1 (PD-L1). In some embodiments, the present invention features a topical composition for skin protection, the composition comprising a small molecule antagonist of programmed death-ligand 1 (PD-L1) comprising BMS-202 or derivatives thereof. In other embodiments, the present invention features a topical composition for skin protection, the composition comprising a BMS-202 prodrug.

The present invention may also feature compositions for use in a method of suppressing immunotoxic effects caused by solar radiation, the composition comprising a small molecule antagonist of programmed death-ligand 1 (PD-L1).

The present invention may further feature a composition for use in a method of treating skin cancer, the composition comprising a small molecule antagonist of programmed death-ligand 1 (PD-L1). In some embodiments, the present invention features a topical composition for use in a method of treating skin cancer, the topical composition comprising a small molecule antagonist of programmed death-ligand 1 (PD-L1). In further embodiments, the present invention features a topical composition for use in a method of treating skin cancer, the topical composition comprising a small molecule antagonist of programmed death-ligand 1 (PD-L1) comprising BMS-202 or derivatives thereof.

### EXAMPLE

The following is a non-limiting example of the present invention. It is to be understood that said example is not intended to limit the present invention in any way. Equivalents or substitutes are within the scope of the present invention.

### Human Specimens, Immunohistochemistry, and Reverse-Phase Proteomics Microarray.

Human tumor samples and skin biopsies were obtained. Samples were classified as normal sun-protected skin (n=20), low-risk (n=39) or high-risk (n=30) cSCC using clinical evaluation and verification from pathological analysis according to the Brigham and Women's Hospital staging system. Immunohistochemical (IHC) staining of human skins for PD-L1 was performed using the SP263 kit (Ventana Medical Systems). Staining of tissue sections was measured using ImageProPlus (Media Cybernetics), a Leica DMR microscope and a Sony 3CCD color video camera. For all tissues (epidermis or cSCC), analysis was confined to keratinocytic cells. For human PD-L1, IHC tissue sections were independently scored by a dermatopathologist to determine the percentage of epidermis or tumor tissue staining positive for PD-L1, with a cutoff of >5% per field qualifying positive staining. For each marker, the percent positive cytoplasmic area (40× field) was determined per tissue specimen (averaging three fields). IHC images shown are magnified according to the 100 μm scale bar contained in the respective panel as specified throughout the figure legends. Statistical significance of differences in PD-L1 expression (>5% between sun-protected, low-risk, and high-risk cSCC) was tested using Kruskal-Wallis non-parametric data analysis (GraphPad Prism 10.0 software).

A second set of human skin biopsies, confirmed by a dermatopathologist to be ‘normal’ (sun-protected; SP, buttock, n=21) or ‘sun-damaged’ (SD; forearm skin, n=10 each for mild and moderate; n=8 for severe) used macrodissection to separate the dermis from the epidermis. PD-L1 was quantified in epidermal lysates using proteomic analysis via reverse phase protein microarray (RPPA) as previously described. Briefly, RPPA was constructed using a 2470 Aushon Arrayer (Aushon BioSystems) equipped with 185 μm pins, and samples were immobilized onto nitrocellulose-coated glass slides (Grace Biolabs) in technical replicates (n=3). PD-L1 levels were measured using a commercially available tyramide-based Catalyzed Signal Amplification System (CSA, Dako) coupled with a fluorescent streptavidin-conjugated IRDye680 dye. Antibody and Sypro Ruby Protein Blot stained arrays were scanned with a laser PowerScanner (TECAN) using the appropriate wavelength channel. Image analysis was performed using commercially available software (MicroVigene v5.1.0.0, VigeneTech, Inc.). The software automatically performs spot finding and subtraction of local background and unspecific signals. Samples were then normalized to the amount of protein and averaged across replicates. Log₂-transformed expression levels of PD-L1 (clone E1L3N) in the epidermis were shown by waterfall plot and box-and-whisker plots for the four groups of ‘sun-protected’ (SP) skin and mild, moderate, and severe ‘sun-damaged’ (SD) skin samples. The two groups of SP and all SD samples were compared using generalized estimating equations to account for the potential correlation of expression levels in SP and SD samples from the same patient.

### Mouse Irradiation

UVB exposure was performed on AP-1 luciferase reporter mice on the SKH-1 genetic background at a dose of 2.75 kJ/m2 using FS40 bulbs (Q-Lab Corporation) as described before with the modification that mice were imaged 24 hr post UVB exposure using whole body bioluminescence instead of 48 hr post UVB using ear punches. SSL irradiation of SKH-1 mice (Charles River Laboratories, strain code 477) at a dose of 90 kJ/m2 UVA/6 kJ/m2 UVB was performed using UVA-340 bulbs (Q-Lab Corporation) as described before. No sex differences were noted in UV-induced PD-L1 cutaneous responses, as published before, and most experiments displayed were conducted in male mice.

### AP-1 SKH-1 Reporter Mouse Bioluminescence

Male transgenic SKH-1 mice heterozygous for the TPA-Response Element (TRE)-driven luciferase transgene (“AP-1 luciferase” mice, n=3) were treated topically on their backs with 200 μL of 8 mM BMS-202 or vehicle control (acetone). For all mice receiving BMS-202, the compound was applied twice before (24 hr and 1 hr) as well as immediately after UV exposure, a dose regimen ensuring efficient target modulation, similar to topical non-sunscreen photoprotection approaches. Mice were injected i.p. with luciferin (potassium salt) stock solution in PBS without magnesium or calcium at a dose of <150 mg/kg of body weight 24 hr after UV exposure and imaged (Lago instrument, Spectral Instruments Imaging) with Aura software analysis.

### AP-1 Reporter Luciferase Assay in HaCaT Keratinocytes

HaCaT cells stably transfected with the TRE-driven luciferase plasmid (“AP-1 luciferase” cells) were seeded in 6-well plates, grown to 70% confluence and serum starved overnight to reduce background signaling. Cells were pretreated with BMS-202 or vehicle (DMSO) for 1 hr, then washed twice with PBS prior to exposure to 250 kJ/m2 UVB. Cells were then washed once more with PBS before being placed into fresh starvation media with vehicle or BMS-202 until harvest 12 hr later. Cells were lysed in Promega's Cell Culture Lysis Buffer (1×), and a total of 10 μg protein per sample replicate was assayed for luciferase activity according to the manufacturer's instructions for the Luciferase Assay System (Promega) using a TD 20/20 luminometer (Turner Designs). Experimental triplicates were averaged and the means from each independent experiment were analyzed by Student's t-test for statistical significance. Results are representative of three independent experiments.

### RT-qPCR Gene Expression Analysis.

Total RNA was isolated from SSL-treated mouse skin using the Qiagen RNeasy Mini Kit (Qiagen) according to the manufacturer's protocol. RNA integrity was checked by the RNA 6000 Nano chip kit using Agilent 2100 Bioanalyzer (Agilent Technologies). Mouse 20× primer/probes [II6 (Mm_00446190_m1), II0 (Mm_01288386_m1), II1b (Mm_00434228_m1), Tnf (Mm_00443258_m1), Tlr4 (Mm_00445273_m1), Ptgs2 (Mm_00478374_m1), Pdl1 (Mm_00452054_m1), Rps18 (housekeeping gene; Mm_02601777_g1)] were obtained from ThermoFisher Scientific. 500 ng of total RNA was used for cDNA synthesis using the following cycling conditions: 25° C. for 10 min; 48° C. for 30 min, and 95° C. for 5 min performed in MJ Thermocycler PTC-200 (MJ Research). Then, 10 ng of cDNA was used for amplification of target genes by quantitative PCR using the following conditions: 95° C. for 10 min followed by 95° C. for 15 sec and 60° C. for 1 min for a total of 40 cycles performed in the ABI7500 Real-Time PCR System (Applied Biosystems). PCR amplification of the human housekeeping gene RPS18 was used to control the quality of the cDNA. Non-template controls were included on each PCR plate. Expression levels of target genes were normalized to the RPS18 control [ΔCt=Ct (gene of interest)−Ct (housekeeping gene)]. After amplification plots were generated, the Ct values (cycle number at which fluorescence reaches threshold) were recorded and quantified using the comparative (ΔΔCt) Ct method as described in the ABI Prism 7500 sequence detection system user guide. Statistical significance was calculated employing the Student's two-tailed t-test.

### Mouse Immunohistochemistry

Caspase-3: Briefly, deparaffinized mouse skin slides were subjected to antigen retrieval using a Decloaking chamber in Rodent Decloaker HIER solution (both from Biocare, 115° C. 30 seconds, then 90° C. 10 seconds) followed by blocking with first 3% hydrogen peroxide (10 minutes) and then 5% normal goat serum in PBST (1 hour). Slides were then incubated in a 1:300 dilution of anti-cleaved caspase-3 antibody (#9661, Cell Signal Technology) at 4° C. overnight. Detection utilized a Vectastain ABC immunoperoxidase kit (PD-6100) with a biotinylated anti-rabbit IgG secondary antibody (Vector Laboratories, BA-1000) and NovaRED substrate according to manufacturer's directions. Negative control slides were exposed to secondary antibodies only and displayed no detectable staining (data not shown). Slides were counterstained in dilute Hematoxylin Gill III for 2 seconds (Leica Biosystems). For quantification, the average count (caspase 3-positive per total epidermal cells) in four fields/slide (20× field) was determined for each mouse skin specimen and calculated per group (n=3).

PD-L1: IHC staining for PD-L1 in mouse skins was performed using the NBP1-43262 antibody (Novus). Epidermal PD-L1 expression is shown as percent staining determined by ImageJ (NIH, open source) quantification using color deconvolution of the brown stain with a uniform threshold between all samples. Each skin sample (n=3, 20× field) was used to measure at least 3 fields per specimen with the exclusion of hair follicles, if present.

Mouse IHC data were analyzed using Kruskal-Wallis non-parametric data analysis (GraphPad Prism 10.0 software). IHC images shown are magnified according to the 100 μm scale bar contained in the respective panel as specified throughout the figure legends.

NanoString nCounter® Gene Expression Analysis

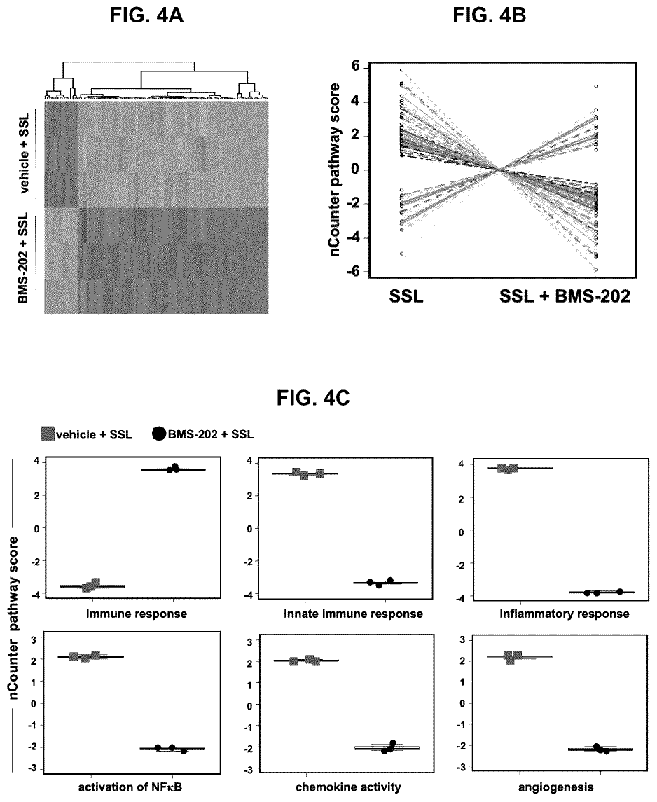

SKH-1 mouse skin [full thickness, 3 biological samples (i.e., 3 mice per treatment group)] treated either with vehicle (acetone) or 8 mM BMS-202 (all 3 times: 24 hr pre, 1 hr pre, and immediately post) and harvested 24 hr after acute SSL. First, total mRNA was prepared using the RNeasy Mini kit (Qiagen). Next, 100 ng was used for NanoString® nCounter® analysis (using the ‘Mouse Inflammation V2’ panel; probing 254 genes, NanoString® Technologies) comparing gene expression between treatment groups. Total mRNA was hybridized with the ‘Mouse Inflammation V2’ code set at 65° C. overnight. Further purification and binding of the hybridized probes to the optical cartridge was performed on the nCounter® Prep Station, and finally, the cartridge was scanned on the nCounter® Digital Analyzer. RCC files were then imported into nSolver4.0 software (NanoString® Technologies) and checked for data quality using default QC settings; all samples passed data quality QC. All samples were normalized using the geometric mean of the housekeeper genes. Expression ratios were calculated by dividing the mean values of all samples in one experimental group (UV+BMS-202) by the mean values of all samples in the reference group (UV+vehicle control). For data analysis, low count threshold value was 200. For heatmap depiction (FIGS. 3A and 3B, FIG. 5 and FIG. 6), a z-score for a specific gene indicates the number of SDs away from the mean of expression in the reference samples. For ‘pathway score analysis’ (FIGS. 4A, 4B, and 4C), each sample's gene expression profile was then condensed into a small set of pathway scores using nCounter® Advanced Analysis software (version 2.0.115). Pathway scores were fit using the first principal component of each gene set's data, oriented such that each pathway score has positive weights for at least half its genes. A ‘covariate plot’ displays selected pathway scores against the covariate chosen (i.e., BMS-202 treatment). Numerical pathway score represents the average fold expression change for all genes associated with the specific pathway, with positive scores indicating enhancement and negative scores indicating attenuation; scores are displayed on the same scale via a Z-transformation.

Individual samples were run in triplicate format of biological replicates, and data analysis was performed using the nSolver analysis software (4.0). For p-value adjustment (Benjamini-Yekutieli False Discovery Rate; p-value threshold: 0.05), nCounter® Advanced Analysis software (version 2.0.115) was used. Nonparametric data analysis of murine experimentation was performed using the Mann-Whitney test. Differences between groups were considered significant at *p<0.05.

### Cell Culture and Solar-Simulated UV-Light (SSL) Treatment

Human HaCaT immortalized keratinocytes were maintained in DMEM with 10% FBS and 1× penicillin/streptomycin. Cells were authenticated using STR profiling and tested for mycoplasma regularly. Cells were seeded onto 60 cm dishes at a density of ~200 k/dish and grown for 3 days. Prior to treatment, cells were serum starved overnight to enhance the UV responsiveness of signaling. Cells were ~80% confluent at the time of treatment. BMS-202 (10 μM) in DMSO was added to the media 1 hr prior to SSL (pretreatment).

For SSL treatment, UVA-340 bulbs (Q-Lab Corporation) were used. Before exposure, cells were washed with 1×PBS and then incubated in 4 mL PBS (with 0.01% MgCl2 and 0.01% CaCl2) during irradiation. Cells were exposed to 40 kJ/m2 UVA/2.68 kJ/m2 UVB, then rinsed once more with PBS prior to being placed back into DMEM with 1% FBS+penicillin/streptomycin and vehicle or BMS-202 (post-treatment) and incubated until harvest 24 hr later. Control cells underwent identical processing, except were held in the biosafety cabinet without UV exposure.

Adult human epithelial keratinocytes (HEKs) were purchased from Thermo Scientific and maintained in EpiLife media on collagen-coated plates as per the vendor's instructions. Cells were treated with BMS-202 and SSL as described for HaCaT cells.

### Immunoblot Analysis (Cell Culture and Mouse Epidermis)

Cells were lysed in RIPA containing 1×HALT protease+phosphatase inhibitor cocktail (ThermoFisher Scientific) and 100 mM PMSF and protein concentrations were assessed using BCA assay (Bio-Rad). Mouse epidermal protein lysates for immunoblot analysis were derived from scraped frozen SKH-1 skins as described previously (Blohm-Mangone et al., 2018). Twenty μg of protein/lane were loaded onto 10% gels for electrophoresis/immunoblotting using established protocols. Human cell lysate blots were probed with Cell Signaling PD-L1 antibody #13684, and mouse epidermal lysate blots were probed with Invitrogen PD-L1 antibody #PA5-20343. Beta-actin (Cell Signaling #4970) was used as a loading control. An anti-rabbit secondary antibody (Cell Signaling #7074) was used for all blots using standard chemiluminescent protocols (Thermo Scientific Pico ECL #34577). Densitometric analysis of band intensity was performed using ImageJ (NIH, open source).

PD-L1 Expression is Significantly Increased in Human cSCC as Well as Sun-Damaged Epidermis Compared to Sun-Protected Skin.

First, differential PD-L1 expression in human skin comparing normal skin to cSCC was examined by immunohistochemical (IHC) analysis using an in-house library of banked clinically annotated specimens. IHC of PD-L1 in normal sun-protected skin was compared to that from samples of low-risk or high-risk cSCCs. Determination of cSCC risk status was defined by pathological micro-anatomical assessment of each tumor specimen. Epidermal or tumor percent staining was scored by a board-certified dermatopathologist (FIGS. 1A and 1B). While normal human epidermis shows no PD-L1 expression, low-risk and high-risk cSCCs significantly upregulated this immune checkpoint protein.

In order to test whether PD-L1 upregulation by UV exposure occurs early in the progression from normal skin to cSCC, human biopsies of chronically sun-damaged skin were also examined. IHC of clinically assessed sun-damaged skin samples revealed a mild upregulation of PD-L1 detectable in basal keratinocytes of the epidermis (FIG. 1C). Further analysis of epidermal lysates from a second set of skin samples was performed using reverse phase protein microarray (RPPA) to more quantitatively measure PD-L1 protein expression changes. PD-L1 analysis included biopsies from clinically assessed sun-protected (SP), mild, moderate, or severe sun-damaged (SD) skin. A waterfall plot depiction of these results illustrates the clustering of the majority of SP samples characterized mostly by low PD-L1 expression, whereas the SD samples exhibited higher PD-L1 expression (FIG. 1D, left). Box-and-whisker plot depiction indicates that SD epidermis expresses significantly more PD-L1 than SP epidermis (FIG. 1D, right, p<0.0001), whereas PD-L1 expression between SD subgroups did not differ significantly. Furthermore, analysis of additional samples derived from the same donor set, exposed to acute SSL (2 MED) and then RPPA probed for PD-L1 expression, revealed statistically significant PD-L1 upregulation in response to UV that occurred irrespective of SP or SD status (data not shown).

### The PD-L1 Antagonist BMS-202 Blocks UV-Induced AP-1-Responsive Stress Signaling, Expression of Inflammatory Mediators, and Apoptosis in Mouse Skin.

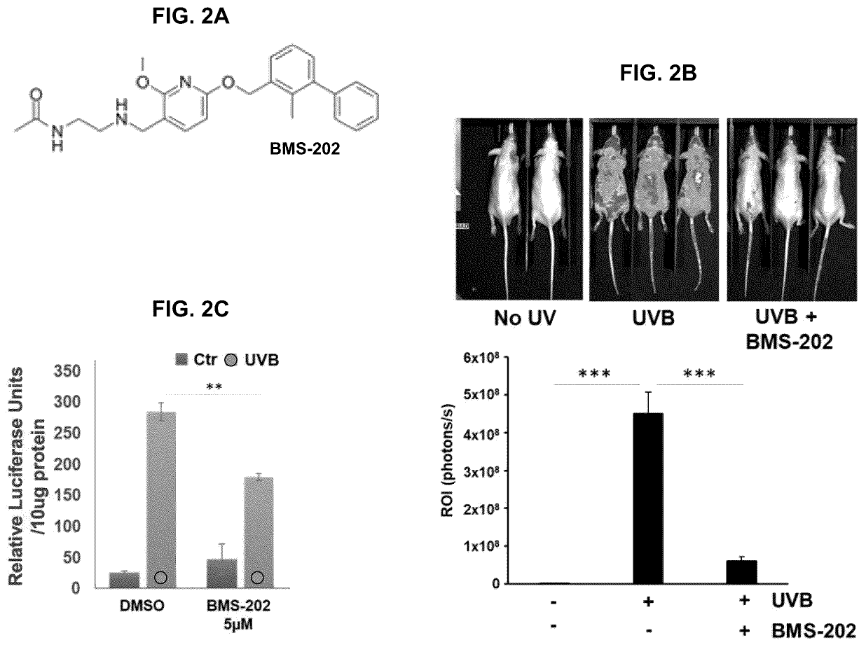

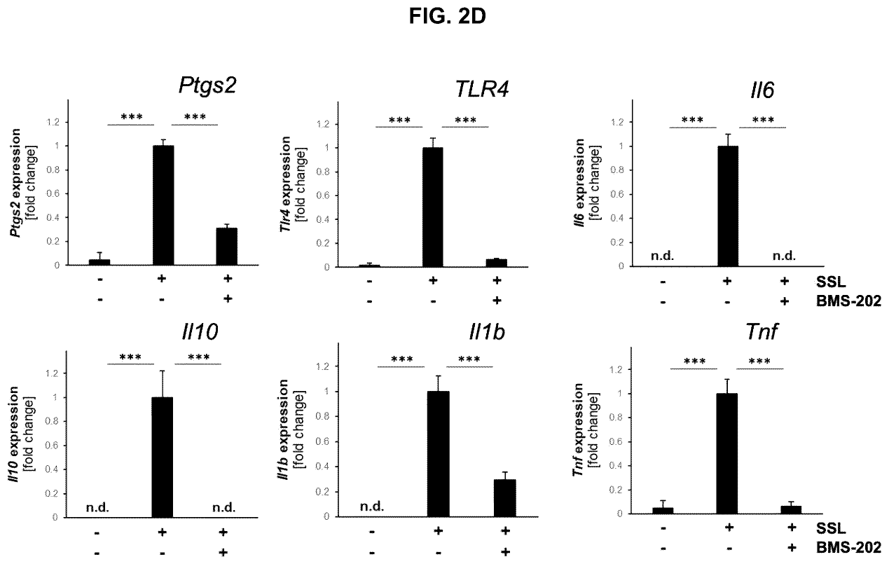

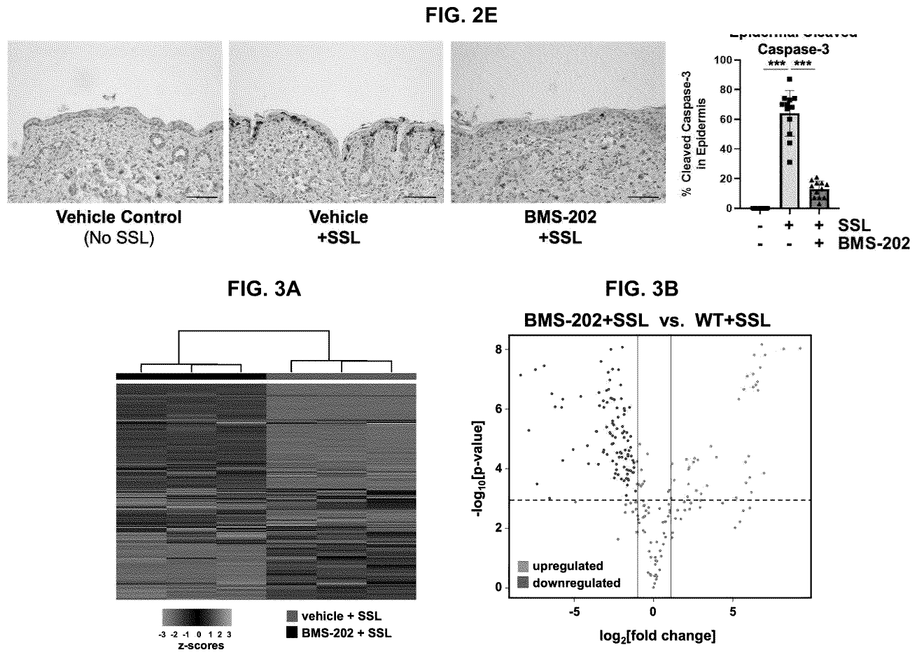

As the above sun-damaged skin indicates that PD-L1 upregulation may occur early in the etiology of skin cancer progression, whether topical pharmacological inhibitors of PD-L1 were assessed to determine if they could affect UV responses in the skin. Among numerous small molecule PD-L1 antagonists available commercially, after screening based on physicochemical properties [absence of UVA/UVB absorbance (data not shown), favorable Log P (3.6), minimal systemic availability upon topical application, lack of cellular toxicity and prior systemic use in animal models] BMS-202 was selected as a potent non-peptidic inhibitor to examine how blocking the activity of PD-L1 would affect skin UV responses (FIG. 2A).

The AP-1 transcription factor plays a part in normal cellular metabolism but is also stimulated in response to cellular stressors, including environmental UV exposure. Notably, PD-L1 is regulated by several transcription factors and signaling pathways known to be stimulated by UV, including AP-1. Transgenic mice, which harbor a ubiquitously expressed luciferase reporter gene under the control of the AP-1 transcription factor-driven TPA-Response Element (the TRE) were maintained. This transgene has been bred onto the outbred SKH-1 hairless immunocompetent mouse line and displays a luciferin-dependent bioluminescent response when exposed to UV light. This model is a valuable tool for testing topical agents in vivo for their stimulatory or inhibitory effects on stress responses in the skin. Therefore, these AP-1 reporter mice were treated topically with either vehicle or BMS-202, with or without subsequent UVB exposure. Mice were imaged for bioluminescence using luciferin injection 24 hr later. Notably, BMS-202 significantly inhibited the UV-induced AP-1 related inflammatory stress response, causing an almost 5-fold attenuation in bioluminescent signal intensity (FIG. 2B). This response was also confirmed in vitro using HaCaT human keratinocytes stably transfected with the AP-1-responsive luciferase construct, which also showed significant inhibition of UVB-induced signal intensity as a consequence of BMS-202 treatment (FIG. 2C).

Following the above AP-1 luciferase assays, expression of select inflammatory and immune-related genes was also probed by independent RT-qPCR analysis of SKH-1 mouse skin which had been treated with vehicle+SSL or 8 mM BMS-202+SSL and harvested 24 hr later. These analyses included cytokines (II1β, II6, II10, Tnfα), and other inflammatory mediators (Ptgs2, Tlr4), all of which displayed strong downregulation of UV-induced expression as a result of BMS-202 treatment (FIG. 2D).

Next, SSL-induced induction of epidermal cell death was probed for by immunohistochemical staining for cleaved caspase-3, an established marker of UV-induced apoptotic cell death observable in SKH-1 mouse skin. As expected, staining intensity was negligible in control samples, while exposure to vehicle+SSL displayed dramatic stimulation of cleaved caspase-3 at 24 hr. Remarkably, treatment with BMS-202 led to a significant reduction in epidermal positivity for this apoptotic marker, reducing the percentage of epidermal cells staining positive for cleaved caspase-3 from over 60% to approximately 10% (FIG. 2E).

### Topical BMS-202 Treatment Suppresses UV-Associated Gene Expression Changes in SKH-1 Mouse Skin as Identified by NanoString® Transcriptomic Analysis.

Next, in order to more broadly assess the consequences of topical BMS-202 treatment on gene expression in UV-exposed SKH-1 mouse skin, NanoString® nCounter® analysis using the nCounter® ‘Mouse Inflammation V2’ panel (probing 254 genes for focused screening of the inflammation and immune response including 6 internal reference controls) was performed. To this end, SKH-1 mice were treated topically with 8 mM BMS-202 or carrier and exposed to SSL as described herein and comparative NanoString expression analysis was performed 24 hr after irradiation. Overall expression analysis employing heatmap (z-score) and volcano plot depictions show significant changes between the treatment groups (FIG. 3B).

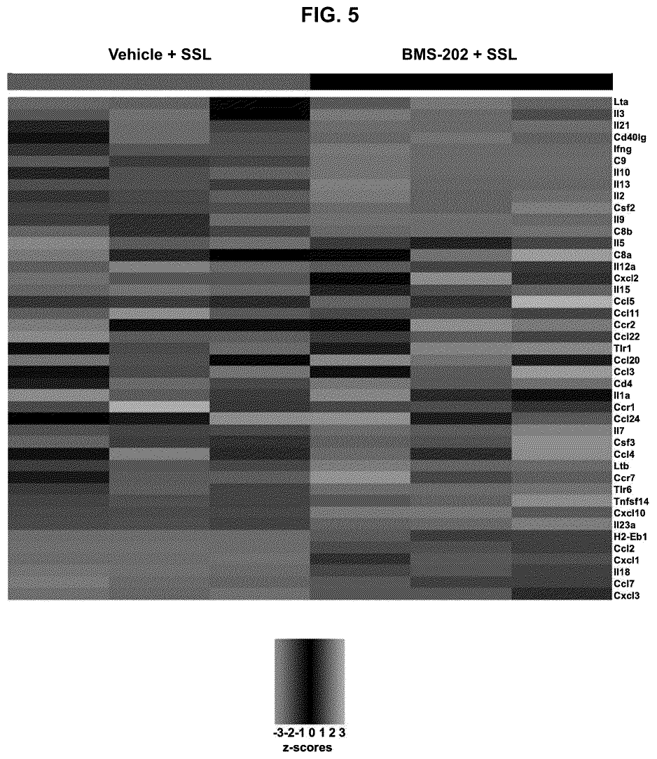

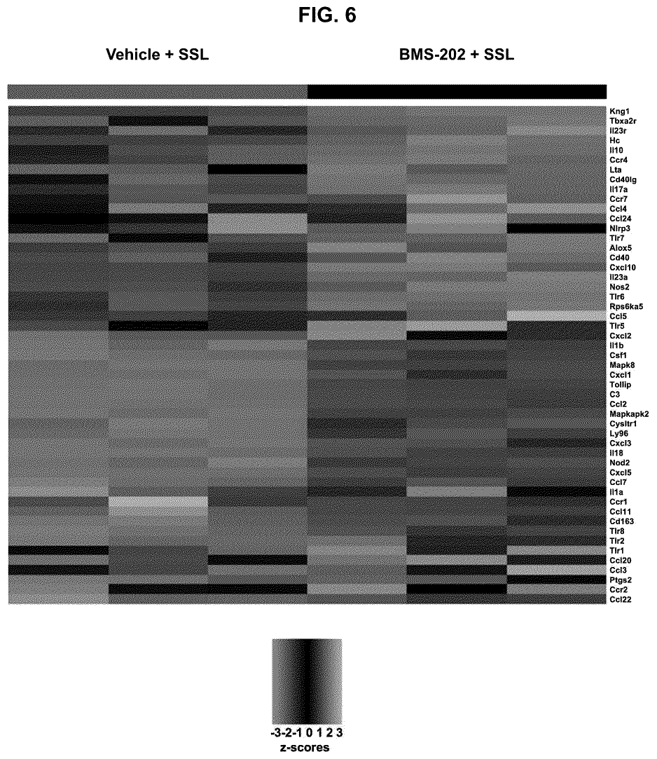

Clustered analysis of statistically significant expression changes indicated that 75 genes displayed BMS-202-responsiveness in SKH-1 mouse skin [>4 fold differential versus vehicle control (upregulated: 23; downregulated: 52); tabular summary: Tables 1 and 2]. Using nCounter® Advanced Analysis software, overall (FIGS. 4A and 4B) and single pathway score profiling identified specific UV-responsive gene expression networks differentially modulated as a function of BMS-202 treatment (FIG. 4C). Remarkably, major pathways known to be activated in the skin upon exposure to solar UV were downregulated in response to BMS-202 including ‘activation of NFκB,’ ‘innate immune response,’ ‘chemokine activity,’ ‘inflammatory response,’ and ‘angiogenesis.’ In contrast, ‘immune response’, known to be antagonized by solar UV exposure, was upregulated in UV-exposed skin as a consequence of BMS-202 treatment (FIG. 4C). Expression heatmaps including gene identity assigned to the ‘inflammatory response’ and ‘immune response’ by nCounter® pathway analysis are depicted in FIG. 5 and FIG. 6 respectively.

Consistent with these pathway score analyses, downregulated expression in response to BMS-202 treatment impacted genes (fold downregulation) in the following domains, among others: Stat signaling [Stat1 (2.8), Stat2 (2.5), Stat3 (7.4)]; AP1 signaling [Fos (4.1), Jun (5.9)]; other transcription factors [Myc (3.3), Nf2el2 (3.5), Hif1a (9.6), Nfatc3 (4.1)]; MAPK signaling [Raf1 (5.7), Rac1 (7.3), Gnas (11.5), Gnb1 (5.8), Mapk8 (3.1), Mapk1 (3.7), Map3k7 (3.7), Map3k1 (4.5), Mapkapk2 (5.1), Map2k1 (5.8), Mapk14 (5.9), Mapk3 (6.3), Map3k5 (6.4), Map2k4 (6.4)]; inflammation and tissue remodeling [Nfkb1 (5.9), Mmp9 (8.2), Mmp3 (3.8), Ptgs1 (3.1), Ptgs2 (1.7), Tgfb1 (13.3), Tgfb3 (2.7); Pdgfa (5.2)]; innate immune signaling [Tlr2 (2.4), Tlr8 (3.4), Myd88 (2.7), Ly96 (coreceptor of Tlr4; 11.0)]; chemokine and cytokine signaling [Il1b (2.9), II1r1 (5.5), Il1rap (3.6), Il6ra (3.4), Il10rb (3.2), Il18 (20.0), Cxcl1 (4.1), Cxcl5 (11.6), Ccl7 (8.1), Ccl2 (5.3), Ccl3, Ccl8 (5.0), Il6ra (3.4)] (Table 1).

TABLE 1
Genes downregulated in BMS-202 + SSL skin relative to SSL
control (NanoString analysis; fold change with adjusted p-values)

| | | | fold | |
|---|---|---|---|---|
| gene | gene ID | gene name | change | p-value |
| Irf1 | 16362 | interferon regulatory factor 1 | −4.1 | 1.47E−06 |
| Nfatc3 | 18021 | nuclear factor of activated T cells, calcineurin | −4.1 | 2.50E−05 |
| | | dependent 3 | | |
| Cxcl1 | 14825 | chemokine (C-X-C motif) ligand 1 | −4.1 | 6.68E−06 |
| Fos | 14281 | FBJ osteosarcoma oncogene | −4.1 | 1.95E−05 |
| Oas1a | 246730 | 2′-5′ oligoadenylate synthetase 1A | −4.3 | 1.71E−05 |
| Tcf4 | 21413 | transcription factor 4 | −4.4 | 7.84E−06 |
| Trem2 | 83433 | triggering receptor expressed on myeloid | −4.5 | 7.51E−07 |
| | | cells 2 | | |
| C1qa | 12259 | complement component 1, q subcomponent, | −4.5 | 2.91E−06 |
| | | alpha | | |
| Grb2 | 14784 | growth factor receptor bound protein 2 | −4.5 | 1.30E−04 |
| Map3k1 | 26401 | mitogen-activated protein kinase kinase | −4.5 | 2.21E−04 |
| | | kinase 1 | | |
| Cdc42 | 12540 | cell division cycle 42 | −4.6 | 7.29E−09 |
| Tyrobp | 22177 | TYRO protein tyrosine kinase binding protein | −4.9 | 7.68E−08 |
| Nr3c1 | 14815 | nuclear receptor subfamily 3, group C, | −4.9 | 1.08E−05 |
| | | member 1 | | |
| Ccl8 | 20307 | chemokine (C-C motif) ligand 8 | −5.0 | 8.38E−06 |
| Nod2 | 257632 | nucleotide-binding oligomerization domain | −5.1 | 1.15E−04 |
| | | containing 2 | | |
| Pdgfa | 18590 | platelet derived growth factor, alpha | −5.2 | 2.52E−07 |
| Mapkapk2 | 17164 | MAP kinase-activated protein kinase 2 | −5.2 | 2.01E−06 |
| Ccl2 | 20296 | chemokine (C-C motif) ligand 2 | −5.3 | 1.22E−07 |
| Mrc1 | 17533 | mannose receptor, C type 1 | −5.4 | 2.24E−06 |
| Il1r1 | 16177 | interleukin 1 receptor, type I | −5.5 | 5.11E−06 |
| Raf1 | 110157 | v-raf-leukemia viral oncogene 1 | −5.7 | 7.03E−06 |
| Gnb1 | 14688 | guanine nucleotide binding protein (G | −5.8 | 1.42E−07 |
| | | protein), beta 1 | | |
| Map2k1 | 26395 | mitogen-activated protein kinase kinase 1 | −5.8 | 5.49E−08 |
| Mapk14 | 26416 | mitogen-activated protein kinase 14 | −5.9 | 2.33E−06 |
| Nfkb1 | 18033 | NF-kappaB1, p50/p105 | −5.9 | 2.59E−06 |
| Rhoa | 11848 | ras homolog family member A | −5.9 | 5.95E−07 |
| Jun | 16476 | jun proto-oncogene | −5.9 | 1.97E−06 |
| C4a | 625018 | complement component 4A | −5.9 | 4.41E−07 |
| Mapk3 | 26417 | mitogen-activated protein kinase 3 | −6.3 | 2.30E−07 |
| Map3k5 | 26408 | mitogen-activated protein kinase kinase | −6.4 | 2.86E−05 |
| | | kinase 5 | | |
| Map2k4 | 26398 | mitogen-activated protein kinase kinase 4 | −6.4 | 3.72E−08 |
| Rac1 | 19353 | Rac family small GTPase 1 | −7.3 | 1.92E−08 |
| Shc1 | 20416 | src homology 2 domain-containing | −7.4 | 1.09E−05 |
| | | transforming protein C1 | | |
| Stat3 | 20848 | signal transducer and activator of | −7.4 | 6.97E−09 |
| | | transcription 3 | | |
| Ccl7 | 20306 | chemokine (C-C motif) ligand 7 | −8.1 | 2.23E−04 |
| Itgb2 | 16414 | integrin beta 2 | −8.2 | 1.52E−06 |
| Mmp9 | 17395 | matrix metallopeptidase 9 | −8.2 | 4.35E−06 |
| Cebpb | 12608 | CCAAT/enhancer binding protein (C/EBP), | −8.3 | 2.18E−06 |
| | | beta | | |
| C3 | 12266 | complement component 3 | −9.0 | 2.68E−07 |
| Cxcl3 | 330122 | chemokine (C-X-C motif) ligand 3 | −9.1 | 2.48E−05 |
| Hif1a | 15251 | hypoxia inducible factor 1, alpha subunit | −9.6 | 2.24E−08 |
| Cfl1 | 12631 | cofilin 1, non-muscle | −10.8 | 3.04E−09 |
| Ly96 | 17087 | lymphocyte antigen 96 | −11.0 | 3.59E−05 |
| Gnas | 14683 | guanine nucleotide binding protein, alpha | −11.5 | 1.31E−06 |
| | | stimulating | | |
| Cxcl5 | 20311 | chemokine (C-X-C motif) ligand 5 | −11.6 | 1.65E−07 |
| Ppp1r12b | 329251 | protein phosphatase 1, regulatory subunit | −12.1 | 1.54E−05 |
| | | 12B | | |
| Bcl6 | 12053 | B cell leukemia/lymphoma 6 | −12.8 | 2.85E−05 |
| Tgfb1 | 21803 | transforming growth factor, beta 1 | −13.3 | 2.02E−05 |
| H2-Eb1 | 14969 | histocompatibility 2, class II antigen E beta | −14.2 | 5.42E−08 |
| Fxyd2 | 11936 | FXYD domain-containing ion transport | −17.0 | 3.07E−05 |
| | | regulator 2 | | |
| Il18 | 16173 | interleukin 18 | −20.0 | 6.86E−06 |
| Mef2c | 17260 | myocyte enhancer factor 2C | −64.9 | 7.36E−05 |

Genes displaying upregulated expression by at least 4-fold in response to BMS-202 exposure compared to vehicle+SSL included interferon-related immune mediators [Ifna (89.3), Ifnb1 (16.4), Ifng (10.6), Ifi44 (5.2)]; interleukins [Il21 (50.1), Il9 (28.9), Il23a (6.5)]; chemokines [Ccr3 (20.5), Cxcl10 (4.3)]; and other immune factors [Defars1 (51.0), C8b (26.2), C9 (18.6), Mbl2 (15.7), Tlr9 (8.3), Nos2 (6.1), Tnfsf14 (4.8)] (Table 2).

TABLE 2
Genes upregulated in BMS-202 + SSL skin relative to SSL
control (NanoString analysis; fold change with adjusted p-values).

| gene | gene ID | gene name | fold change | p-value |
|---|---|---|---|---|
| Ifna1 | 15962 | interferon alpha 1 | 89.3 | 1.58E−05 |
| Defa-rs1 | 13218 | defensin, alpha, 29 | 51.0 | 9.12E−06 |
| Il21 | 60505 | interleukin 21 | 50.1 | 7.69E−06 |
| Mx1 | 17857 | MX dynamin-like GTPase 1 | 36.2 | 3.37E−06 |
| Il9 | 16198 | interleukin 9 | 28.9 | 3.24E−05 |
| C8b | 110382 | complement component 8, beta | 26.2 | 3.64E−05 |
| | | polypeptide | | |
| Crp | 12944 | C-reactive protein, pentraxin-related | 23.9 | 3.08E−06 |
| Ccr3 | 12771 | chemokine (C-C motif) receptor 3 | 20.5 | 2.98E−05 |
| C9 | 12279 | complement component 9 | 18.6 | 1.81E−05 |
| Ifnb1 | 15977 | interferon beta 1, fibroblast | 16.4 | 3.73E−06 |
| Mbl2 | 17195 | mannose-binding lectin (protein C) 2 | 15.7 | 5.08E−05 |
| Kng1 | 16644 | kininogen 1 | 14.9 | 1.58E−05 |
| Cysltr2 | 70086 | cysteinyl leukotriene receptor 2 | 14.4 | 1.66E−04 |
| Ifng | 15978 | interferon gamma | 10.6 | 5.95E−05 |
| Chi3l3 | 12655 | chitinase-like 3 | 10.5 | 4.86E−07 |
| Tlr9 | 81897 | toll-like receptor 9 | 8.3 | 2.54E−04 |
| Il23a | 83430 | interleukin 23, alpha subunit p19 | 6.5 | 3.23E−04 |
| Nos2 | 18126 | nitric oxide synthase 2, inducible | 6.1 | 8.12E−04 |
| Mx2 | 17858 | MX dynamin-like GTPase 2 | 5.3 | 3.00E−04 |
| Ifi44 | 99899 | interferon-induced protein 44 | 5.2 | 3.77E−03 |
| Tnfsf14 | 50930 | tumor necrosis factor superfamily, | 4.8 | 3.15E−03 |
| | | member 14 | | |
| Cxcl10 | 15945 | chemokine (C-X-C motif) ligand 10 | 4.3 | 4.06E−04 |
| Tslp | 53603 | thymic stromal lymphopoietin | 4.3 | 2.15E−06 |

### BMS-202 Suppresses UV-Induced PD-L1 Expression in Cultured Human Keratinocytes And Mouse Epidermis.

Given the pronounced UV-responsiveness of epidermal PD-L1 expression and the remarkable inhibitory effects of BMS-202 on UV-induced stress responses, next the effects of BMS-202 on UV-induced cutaneous PD-L1 expression were tested.

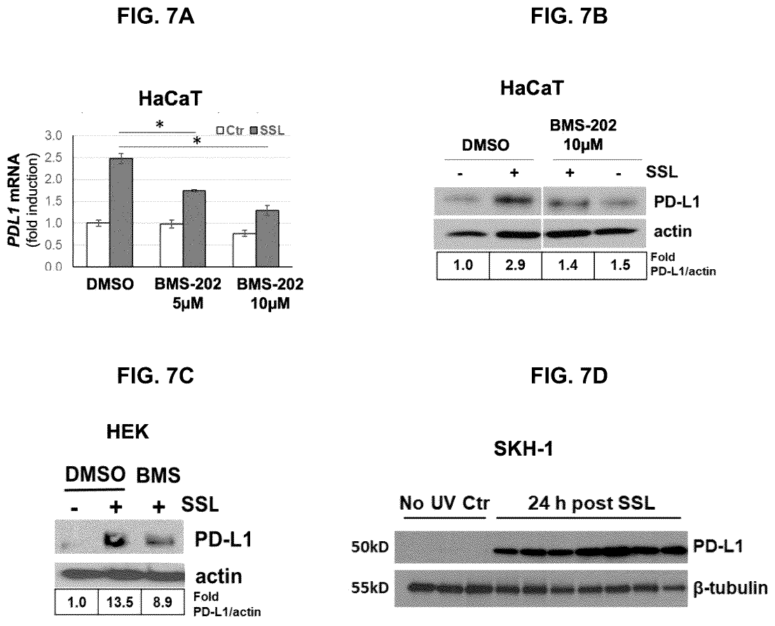

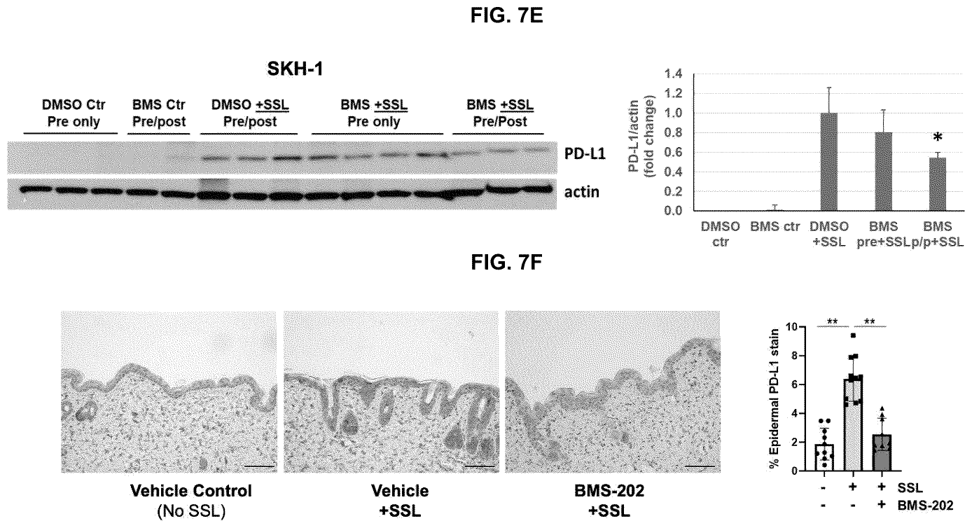

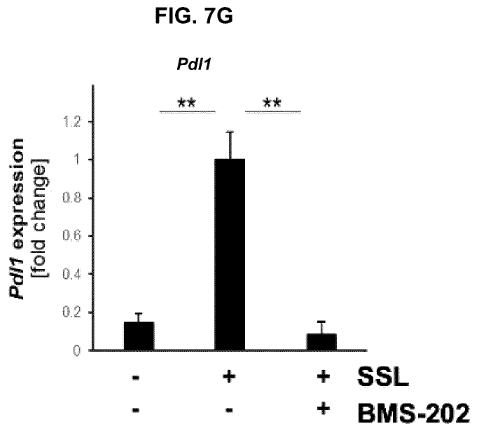

Remarkably, treatment with BMS-202 blocked SSL-induced PD-L1 expression in HaCaT keratinocytes at the mRNA and protein level (FIGS. 7A and 7B), an observation that was reproducible in human primary keratinocytes in culture (HEK cells, FIG. 7C). Immunoblot analysis using SKH-1 mouse epidermis confirms that PD-L1 protein, found at low levels in untreated skin, becomes strongly induced 24 hr post SSL exposure (FIG. 7D). Strikingly, topical BMS-202 significantly antagonized UV-induced PD-L1 upregulation at the protein level in SKH-1 mouse epidermis (FIG. 7E). This antagonistic effect is more pronounced if the compound is applied pre- and post-UV. Importantly, IHC staining for PD-L1 confirms epidermal stimulation of this protein by acute SSL, and its pronounced inhibition by BMS-202 topical application (24 hr post SSL harvest, FIG. 7F). This inhibition also occurs at the transcriptional level as assessed in full thickness mouse skin by RT-qPCR (FIG. 7G).

Based on PD-L1 responsiveness in sun-damaged human skin and the findings that topical BMS-202 application can suppress solar UV-induced AP-1 activation, inflammatory gene expression, PD-L1 upregulation, and caspase-3 cleavage in SKH-1 mouse skin.

### EMBODIMENTS

The following embodiments are intended to be illustrative only and not to be limiting in any way.

Embodiment 1: A method of preventing and reversing damage caused by solar radiation of skin, comprising contacting the skin with a therapeutically effective amount of a composition comprising a small molecule antagonist of programmed death-ligand 1 (PD-L1), thereby protecting the skin from damage caused by solar radiation. Embodiment 2: The method of embodiment 1, wherein the composition is applied topically. Embodiment 3: A method of preventing and reversing damage caused by solar radiation of skin, comprising contacting the skin with a therapeutically effective amount of a topical composition comprising a small molecule antagonist of programmed death-ligand 1 (PD-L1), thereby protecting the skin from damage caused by solar radiation. Embodiment 4: The method of any one of embodiments 1-3, wherein the small molecule comprises BMS-202 or variants thereof. Embodiment 5: A method of preventing and reversing damage caused by solar radiation of skin, comprising contacting the skin with a therapeutically effective amount of a topical composition comprising BMS-202 or variants thereof, thereby protecting the skin from damage caused by solar radiation. Embodiment 6: The method of embodiment 4 or embodiment 5, wherein the variants of BMS-202 are optimized for skin residence time and minimum systemic availability. Embodiment 7: The method of any one of embodiments 3-6, wherein the composition is in a form of a lotion, a cream, a balm, an ointment, a gel, a paste, a spray, a patch, or a solution.

Embodiment 8: The method of any one of embodiments 1-7, wherein the method further reverses and antagonizes damage caused by photodamage, inflammation, an autoimmune disease, carcinogenesis, and pre-cancerous conditions, or photoaging. Embodiment 9: The method of embodiment 8, wherein inflammation comprises contact dermatitis or psoriasis. Embodiment 10: The method of embodiment 8, wherein the autoimmune disease comprises vitiligo, scleroderma, or lupus. Embodiment 11: The method of embodiment 8, wherein the pre-cancerous conditions comprise actinic keratosis or dysplastic nevi.

Embodiment 12: The method of any one of embodiments 1-11, wherein reversing damage occurs by a process of photorejuvenation or photoimmunoprevention. Embodiment 13: The method of any one of embodiments 1-12, wherein the method further prevents the development of precancerous or cancerous states through early molecular interception. Embodiment 14: The method of any one of embodiments 1-13, wherein the solar radiation comprises ultraviolet (UV) rays. Embodiment 15: The method of any one of embodiments 1-14, wherein the method further protects the skin from effects of environmental pollutant exposure. Embodiment 16: The method of embodiment 15, wherein the environmental pollutant comprises ozone, dioxin, smog, particulate matter, benzpyrene, and other polyaromatic hydrocarbons, wherein the particulate matter comprises PM2.5.

Embodiment 17: A method of suppressing immunotoxic effects caused by solar radiation in a subject in need thereof, the method comprising administering a therapeutically effective amount of a small molecule antagonist of programmed death-ligand 1 (PD-L1) to the subject. Embodiment 18: The method of embodiment 17, wherein the small molecule antagonist is administered topically. Embodiment 19: A method of suppressing immunotoxic effects caused by solar radiation in a subject in need thereof, the method comprising administering topically a therapeutically effective amount of a small molecule antagonist of programmed death-ligand 1 (PD-L1) to the subject. Embodiment 20: The method of any one of embodiments 17-19, wherein the small molecule antagonist comprises BMS-202 or variants thereof. Embodiment 21: A method of suppressing immunotoxic effects caused by solar radiation in a subject in need thereof, the method comprising administering topically a therapeutically effective amount of a small molecule antagonist of programmed death-ligand 1 (PD-L1) comprising BMS-202 or variants thereof to the subject. Embodiment 22: The method of any one of embodiments 17-21, wherein the variants of BMS-202 are optimized for skin residence time and minimum systemic availability. Embodiment 23: The method of any one of embodiments 17-22, wherein the small molecule antagonist is in a form of a lotion, a cream, a balm, an ointment, a gel, a paste, a patch, a spray, or a solution. Embodiment 24: The method of any one of embodiments 17-23, wherein the solar radiation comprises ultraviolet (UV) rays.

Embodiment 25: A method of protecting skin, comprising contacting the skin with a therapeutically effective amount of a composition comprising a small molecule antagonist of programmed death-ligand 1 (PD-L1), thereby protecting the skin. Embodiment 26: The method of embodiment 25, wherein the small molecule comprises BMS-202 or variants thereof. Embodiment 27: The method of embodiment 26, wherein the variants of BMS-202 are optimized for skin residence time and minimum systemic availability. Embodiment 28: The method of any one of embodiments 25-27, wherein the composition is administered topically. Embodiment 29: The method of any one of embodiments 25-28, wherein the composition is in a form of a lotion, a cream, a balm, an ointment, a gel, a paste, a patch, a spray, or a solution. Embodiment 30: The method of any one of embodiments 25-29, wherein the method further protects the skin against inflammation, immune dysfunction and immune disturbances, and pre-cancerous conditions and cancer. Embodiment 31: The method of embodiment 30, wherein the pre-cancerous conditions comprise actinic keratosis or dysplastic nevi. Embodiment 32: The method of any one of embodiments 25-31, wherein the method protects the skin against atopic dermatitis, inflammation, and autoimmune disease.

Embodiment 33: A method of preventing or treating skin cancer in a subject in need thereof, the method comprising administering a therapeutically effective amount of a composition comprising a small molecule antagonist of programmed death-ligand 1 (PD-L1). Embodiment 34: The method of embodiment 33, wherein the composition is administered topically. Embodiment 35: A method of preventing or treating skin cancer in a subject in need thereof, the method comprising topically administering a therapeutically effective amount of a composition comprising a small molecule antagonist of programmed death-ligand 1 (PD-L1). Embodiment 36: The method of any one of embodiments 33-35, wherein the small molecule antagonist comprises BMS-202 or variants thereof. Embodiment 37: A method of preventing or treating skin cancer in a subject in need thereof, the method comprising topically administering a therapeutically effective amount of a composition comprising a small molecule antagonist of programmed death-ligand 1 (PD-L1) comprising BMS-202 or variants thereof. Embodiment 38: The method of embodiment 36 or embodiment 37, wherein the variants of BMS-202 are optimized for skin residence time and minimum systemic availability. Embodiment 39: The method of any one of embodiments 33-38, wherein the composition is in a form of a lotion, a cream, a balm, an ointment, a gel, a paste, a spray, a patch, or a solution. Embodiment 40: The method of any one of embodiments 33-39, wherein the skin cancer comprises cutaneous squamous cell carcinoma (cSCC) and basal cell carcinoma (BCC).

Embodiment 41: A topical composition for skin protection, the composition comprising a small molecule antagonist of programmed death-ligand 1 (PD-L1). Embodiment 42: A topical composition for skin protection, the composition comprising a small molecule antagonist of programmed death-ligand 1 (PD-L1) comprising BMS-202 or derivatives thereof. Embodiment 43: A topical composition for skin protection, the composition comprising a prodrug or derivatives of BMS-202. Embodiment 44: The composition of any one of embodiments 41-43, wherein the derivatives are optimized for skin residence time and minimum systemic availability.

Embodiment 45: A composition for use in a method of preventing and reversing damage caused by solar radiation of skin, the composition comprising a small molecule antagonist of programmed death-ligand 1 (PD-L1). Embodiment 46: A topical composition for use in a method of preventing and reversing damage caused by solar radiation of skin, the topical composition comprising a small molecule antagonist of programmed death-ligand 1 (PD-L1). Embodiment 47: A topical composition for use in a method of preventing and reversing damage caused by solar radiation of skin, the topical composition comprising a small molecule antagonist of programmed death-ligand 1 (PD-L1) comprising BMS-202 or derivatives thereof.

Embodiment 48: A composition for use in a method of suppressing immunotoxic effects caused by solar radiation, the composition comprising a small molecule antagonist of programmed death-ligand 1 (PD-L1). Embodiment 49: A topical composition for use in a method of suppressing immunotoxic effects caused by solar radiation, the topical composition comprising a small molecule antagonist of programmed death-ligand 1 (PD-L1). Embodiment 50: A topical composition for use in a method of suppressing immunotoxic effects caused by solar radiation, the topical composition comprising a small molecule antagonist of programmed death-ligand 1 (PD-L1) comprising BMS-202 or derivatives thereof.

Embodiment 51: A composition for use in a method of treating skin cancer, the composition comprising a small molecule antagonist of programmed death-ligand 1 (PD-L1). Embodiment 52: A topical composition for use in a method of treating skin cancer, the topical composition comprising a small molecule antagonist of programmed death-ligand 1 (PD-L1). Embodiment 53: A topical composition for use in a method of treating skin cancer, the topical composition comprising a small molecule antagonist of programmed death-ligand 1 (PD-L1) comprising BMS-202 or derivatives thereof.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1.-2. (canceled)

3. A method of preventing and reversing damage caused by solar radiation of skin, comprising contacting the skin with a therapeutically effective amount of a topical composition comprising a small molecule antagonist of programmed death-ligand 1 (PD-L1), thereby protecting the skin from damage caused by solar radiation.

4. The method of claim 3, wherein the small molecule comprises BMS-202 or variants thereof.

5. (canceled)

6. The method of claim 4, wherein the variants of BMS-202 are optimized for skin residence time and minimum systemic availability.

7. The method of claim 3, wherein the composition is in a form of a lotion, a cream, a balm, an ointment, a gel, a paste, a spray, a patch, or a solution.

8. The method of claim 3, wherein the method further reverses and antagonizes damage caused by photodamage, inflammation, an autoimmune disease, carcinogenesis, and pre-cancerous conditions, or photoaging.

9.-11. (canceled)

12. The method of claim 3, wherein reversing damage occurs by a process of photorejuvenation or photoimmunoprevention.

13. The method of claim 3, wherein the method further prevents the development of precancerous or cancerous states through early molecular interception.

14. The method of claim 3, wherein the solar radiation comprises ultraviolet (UV) rays.

15. The method of claim 3, wherein the method further protects the skin from effects of environmental pollutant exposure.

16. The method of claim 15, wherein the environmental pollutant comprises ozone, dioxin, smog, particulate matter, benzpyrene, and other polyaromatic hydrocarbons, wherein the particulate matter comprises PM2.5.

17.-24. (canceled)

25. A method of protecting skin, comprising contacting the skin with a therapeutically effective amount of a topical composition comprising a small molecule antagonist of programmed death-ligand 1 (PD-L1), thereby protecting the skin.

26. The method of claim 25, wherein the small molecule comprises BMS-202 or variants thereof.

27. The method of claim 26, wherein the variants of BMS-202 are optimized for skin residence time and minimum systemic availability.

28. (canceled)

29. The method of claim 25, wherein the composition is in a form of a lotion, a cream, a balm, an ointment, a gel, a paste, a patch, a spray, or a solution.

30. The method of claim 25, wherein the method further protects the skin against inflammation, immune dysfunction, and immune disturbances, and pre-cancerous conditions and cancer.

31.-34. (canceled)

35. A method of preventing or treating skin cancer in a subject in need thereof, the method comprising topically administering a therapeutically effective amount of a composition comprising a small molecule antagonist of programmed death-ligand 1 (PD-L1).

36. The method of claim 35, wherein the small molecule antagonist comprises BMS-202 or variants thereof.

37. (canceled)

38. The method of claim 36, wherein the variants of BMS-202 are optimized for skin residence time and minimum systemic availability.

39. The method of claim 35, wherein the composition is in a form of a lotion, a cream, a balm, an ointment, a gel, a paste, a spray, a patch, or a solution.

40. The method of claim 35, wherein the skin cancer comprises cutaneous squamous cell carcinoma (cSCC) and basal cell carcinoma (BCC).

41.-53. (canceled)
